(19) United States
(12) Patent Application Publication
(10) Pub. No.: US 2006/0121109 A1
(43) Pub. Date: Jun. 8, 2006

(54) Formulations of substituted benzoxazoles

(75) Inventors: Michael J. Rowley, Fareham (GB); Angela C. Potts, Fareham (GB); Christopher S. Wilson, Gosport (GB); Marc S. Tesconi, Monroe, NY (US); Mannching Sherry Ku, Thiells, NY (US)

Correspondence Address: COZEN O' CONNOR, P. C., 1900 MARKET STREET, PHILADELPHIA, PA, 19103-3508, US

(73) Assignee: Wyeth, Madison, NJ (US)

(21) Appl. No.: 11/290,184

(22) Filed: Nov. 30, 2005

Related U.S. Application Data
(60) Provisional application No. 60/632,448, filed on Dec. 2, 2004.

(51) Int. Cl. A61K 31/428; A61K 31/423; A61K 31/4184; A61K 9/20

(52) U.S. Cl. 424/464; 514/367; 514/375; 514/394

(57) ABSTRACT

The present invention provides solid dosage formulations of benzoxazole-containing ERβ-selective ligands, and processes for their manufacture, more particularly to novel formulations, and processes for their manufacture, that contain the ERβ-selective ligand, ERB-041.

### CROSS-REFERENCE TO RELATED APPLICATION

The present invention claims benefit of priority from provisional U.S. Patent Application Ser. No. 60/632,448 filed Dec. 2, 2004, which is incorporated herein by reference in its entirety.

### FIELD OF THE INVENTION

The present invention relates to solid dosage formulations that include ERβ-selective ligands that contain benzoxazole (or benzothiazole or benzoimidazole), and processes for manufacture of said formulations, more particularly to novel formulations and processes for manufacture of formulations containing the ERβ-selective ligand, ERB-041.

### BACKGROUND OF THE INVENTION

This invention relates to formulations for substituted benzoxazoles (or benzothiazoles or benzoimidazoles), which are useful as estrogenic agents.

The pleiotropic effects of estrogens in mammalian tissues have been well documented, and it is now appreciated that estrogens affect many organ systems [Mendelsohn and Karas, New England Journal of Medicine 340: 1801-1811 (1999), Epperson, et al., Psychosomatic Medicine 61: 676-697 (1999), Crandall, Journal of Women's Health & Gender Based Medicine 8: 1155-1166 (1999), Monk and Brodaty, Dementia & Geriatric Cognitive Disorders 11: 1-10 (2000), Hum and Macrae, Journal of Cerebral Blood Flow & Metabolism 20: 631-652 (2000), Calvin, Maturitas 34: 195-210 (2000), Finking, et al., Zeitschrift fur Kardiologie 89: 442-453 (2000), Brincat, Maturitas 35: 107-117 (2000), Al-Azzawi, Postgraduate Medical Journal 77: 292-304 (2001)]. Estrogens can exert effects on tissues in several ways, and the most well characterized mechanism of action is their interaction with estrogen receptors leading to alterations in gene transcription. Estrogen receptors are ligand-activated transcription factors and belong to the nuclear hormone receptor superfamily. Other members of this family include the progesterone, androgen, glucocorticoid and mineralocorticoid receptors. Upon binding ligand, these receptors dimerize and can activate gene transcription either by directly binding to specific sequences on DNA (known as response elements) or by interacting with other transcription factors (such as AP1), which in turn bind directly to specific DNA sequences [Moggs and Orphanides, EMBO Reports 2: 775-781 (2001), Hall, et al., Journal of Biological Chemistry 276: 36869-36872 (2001), McDonnell, Principles of Molecular Regulation. 351-361 (2000)]. A class of “coregulatory” proteins can also interact with the ligand-bound receptor and further modulate its transcriptional activity [McKenna, et al., Endocrine Reviews 20: 321-344 (1999)]. It has also been shown that estrogen receptors can suppress NFκB-mediated transcription in both a ligand-dependent and independent manner [Quaedackers, et al., Endocrinology 142: 1156-1166 (2001), Bhat, et al., Journal of Steroid Biochemistry & Molecular Biology 67: 233-240 (1998), Pelzer, et al., Biochemical & Biophysical Research Communications 286: 1153-7 (2001)].

Estrogen receptors can also be activated by phosphorylation. This phosphorylation is mediated by growth factors such as EGF and causes changes in gene transcription in the absence of ligand [Moggs and Orphanides, EMBO Reports 2: 775-781 (2001), Hall, et al., Journal of Biological Chemistry 276: 36869-36872 (2001)].

A less well-characterized means by which estrogens can affect cells is through a so-called membrane receptor. The existence of such a receptor is controversial, but it has been well documented that estrogens can elicit very rapid non-genomic responses from cells. The molecular entity responsible for transducing these effects has not been definitively isolated, but there is evidence to suggest it is at least related to the nuclear forms of the estrogen receptors [Levin, Journal of Applied Physiology 91: 1860-1867 (2001), Levin, Trends in Endocrinology & Metabolism 10: 374-377 (1999)].

Two estrogen receptors have been discovered to date. The first estrogen receptor was cloned about 15 years ago and is now referred to as ERα [Green, et al., Nature 320: 134-9 (1986)]. The second form of the estrogen receptor was found comparatively recently and is called ERβ [Kuiper, et al., Proceedings of the National Academy of Sciences of the United States of America 93: 5925-5930 (1996)]. Early work on ERβ focused on defining its affinity for a variety of ligands and indeed, some differences with ERα were seen. The tissue distribution of ERβ has been well mapped in the rodent and it is not coincident with ERα. Tissues such as the mouse and rat uterus express predominantly ERα, whereas the mouse and rat lung express predominantly ERβ [Couse, et al., Endocrinology 138: 4613-4621 (1997), Kuiper, et al., Endocrinology 138: 863-870 (1997)]. Even within the same organ, the distribution of ERα and ERβ can be compartmentalized. For example, in the mouse ovary, ERβ is highly expressed in the granulosa cells and ERα is restricted to the thecal and stromal cells [Sar and Welsch, Endocrinology 140: 963-971 (1999), Fitzpatrick, et al., Endocrinology 140: 2581-2591 (1999)]. However, there are examples where the receptors are coexpressed and there is evidence from in vitro studies that ERα and ERβ can form heterodimers [Cowley, et al., Journal of Biological Chemistry 272: 19858-19862 (1997)].

A large number of compounds have been described that either mimic or block the activity of 17β-estradiol. Compounds having roughly the same biological effects as 17β-estradiol, the most potent endogenous estrogen, are referred to as “estrogen receptor agonists”. Those which, when given in combination with 17β-estradiol, block its effects are called “estrogen receptor antagonists”. In reality there is a continuum between estrogen receptor agonist and estrogen receptor antagonist activity and indeed some compounds behave as estrogen receptor agonists in some tissues and estrogen receptor antagonists in others. These compounds with mixed activity are called selective estrogen receptor modulators (SERMS) and are therapeutically useful agents (e.g. EVISTA®) [McDonnell, Journal of the Society for Gynecologic Investigation 7: S10-S15 (2000), Goldstein, et al., Human Reproduction Update 6: 212-224 (2000)]. The precise reason why the same compound can have cell-specific effects has not been elucidated, but the differences in receptor conformation and/or in the milieu of coregulatory proteins have been suggested.

It has been known for some time that estrogen receptors adopt different conformations when binding ligands. However, the consequence and subtlety of these changes has been only recently revealed. The three dimensional structures of ERα and ERβ have been solved by co-crystallization with various ligands and clearly show the repositioning of helix 12 in the presence of an estrogen receptor antagonist that sterically hinders the protein sequences required for receptor-coregulatory protein interaction [Pike, et al., EMBO 18: 4608-4618 (1999), Shiau, et al., Cell 95: 927-937 (1998)]. In addition, the technique of phage display has been used to identify peptides that interact with estrogen receptors in the presence of different ligands [Paige, et al., Proceedings of the National Academy of Sciences of the United States of America 96: 3999-4004 (1999)]. For example, a peptide was identified that distinguished between ERα bound to the full estrogen receptor agonists 17β-estradiol and diethylstilbesterol. A different peptide was shown to distinguish between clomiphene bound to ERα and ERβ. These data indicate that each ligand potentially places the receptor in a unique and unpredictable conformation that is likely to have distinct biological activities.

The preparation of exemplary ERβ selective ligands, including 2-(3-fluoro-4-hydroxyphenyl)-7-vinyl-1,3-benzoxazol-5-ol (ERB-041), is described in U.S. Pat. No. 6,794,403, incorporated herein by reference in its entirety.

As mentioned above, estrogens affect a panoply of biological processes. In addition, where gender differences have been described (e.g., disease frequencies, responses to challenge, etc.), it is possible that the explanation involves the difference in estrogen levels between males and females.

Given the importance of these compounds as pharmaceutical agents, it can be seen that effective formulations for delivery of the compounds is of great import. This invention is directed to these, as well as other, important ends.

### SUMMARY OF THE INVENTION

In some embodiments, the present invention provides pharmaceutical formulations comprising a pharmaceutically effective amount of an active pharmacological agent and a carrier or excipient system, the carrier or excipient system comprising:

a) a filler/diluent component comprising from about 10% to about 60% by weight of the pharmaceutical formulation;

b) a surface modifying agent component comprising from about 1% to about 20% by weight of the pharmaceutical formulation;

c) a glidant/disintegrant component comprising from about 0.01% to about 10% by weight of the pharmaceutical formulation;

d) an optional second filler/diluent component comprising up to about 20% by weight of the pharmaceutical formulation; and

e) a lubricant component comprising up to about 10% by weight of the pharmaceutical formulation;
wherein the active pharmacological agent has the Formula I: 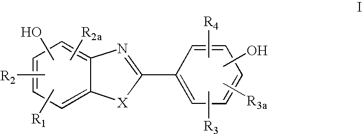
wherein
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —F₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R_{6,} NR₅R₆ or N(R₅)COR₆;
R₅, R₆ are each, independently hydrogen, alkyl of 1-6 carbon atoms, or aryl of 6-10 carbon atoms;
X is O, S, or N R₇; and
R₇ is hydrogen, alkyl of 1-6 carbon atoms, aryl of 6-10 carbon atoms, —COR₅, —CO₂R₅ or —SO₂R₅;
   or a pharmaceutically acceptable salt thereof.

In some embodiments, X is O. In some further embodiments, R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some embodiments, the active ingredient is 2-(3-fluoro-4-hydroxyphenyl)-7-vinyl-1,3-benzoxazol-5-ol or a pharmaceutically acceptable salt thereof.

The term halogen refers to chloro, bromo, fluoro or iodo, preferably fluoro. The alkyl of 1-6 carbon atoms (used alone or as part of a group e.g. alkoxy) may be a straight or branched alkyl e.g. methyl, ethyl, n-propyl, i-propyl or n-butyl. The cycloalkyl of 3-8 carbon atoms may be saturated or unsaturated and includes the moieties cyclopropyl, cyclobutyl, cyclopentyl and cyclohexyl. The trifluoroalkyl of 1-6 carbon atoms (used alone or as part of a group) may suitably be trifluoromethyl. Sulfoxoalkyl of 1-6 carbon atoms refers to the group —SO—R wherein R is an alkyl of 1-6 carbon atoms as defined above. Aryl of 6-10 carbon atoms refers to a mono or poly cyclic aromatic group. e.g., phenyl or napthyl. The 5 to 6 membered heterocyclic ring having 1 to 4 heteroatoms selected from O, N or S is a saturated, partially unsaturated or aromatic ring, e.g., a furanyl, pyranyl, pyridinyl, pyrimidinyl, pyrazinyl, morpholinyl, thiomorpholinyl, imidazolyl, oxazolyl, thioxazolyl, thienyl or piperidinyl ring. The alkynyl of 2-7 carbon atoms is a group having at least one triple bond, e.g., ethynyl. The alkenyl of 2-7 carbon atoms is a group having at least one double bond, e.g., vinyl. When the alkyl or alkenyl moieties are substituted they may be substituted with 1 or more substituents as defined above, e.g. by 1, 2 or 3 substituents which may be the same or different.

In some embodiments, the active pharmacological agent comprises up to about 88% by weight of the pharmaceutical formulation.

In some embodiments, the active pharmacological agent comprises from about 10% to about 50% by weight of the pharmaceutical formulation; the filler/diluent component comprises from about 30% to about 60% by weight of the pharmaceutical formulation; the surface modifying agent component comprises from about 1% to about 10% by weight of the pharmaceutical formulation; the glidant/disintegrant component comprises from about 0.01% to about 5% by weight of the pharmaceutical formulation; the optional second filler/diluent component comprises from about 10% to about 20% by weight of the pharmaceutical formulation; and the lubricant component comprises from about 0.01% to about 2% by weight of the pharmaceutical formulation.

In some further embodiments, the active pharmacological agent comprises from about 20% to about 40% by weight of the pharmaceutical formulation; the filler/diluent component comprises from about 40% to about 60% by weight of the pharmaceutical formulation; the surface modifying agent component comprises from about 3% to about 7% by weight of the pharmaceutical formulation; the glidantidisintegrant component comprises from about 1% to about 2% by weight of the pharmaceutical formulation; the optional second filler/diluent component comprises from about 10% to about 20% by weight of the pharmaceutical formulation; and the lubricant component comprises from about 0.01% to about 1% by weight of the pharmaceutical formulation.

In further embodiments, the active pharmacological agent comprises from about 25% to about 35% by weight of the pharmaceutical formulation; the filler/diluent component comprises from about 44% to about 53% by weight of the pharmaceutical formulation; the surface modifying agent component comprises from about 4% to about 6% by weight of the pharmaceutical formulation; the glidantidisintegrant component comprises from about 1% to about 2% by weight of the pharmaceutical formulation; the optional second filler/diluent component comprises from about 12% to about 18% by weight of the pharmaceutical formulation; and the lubricant component comprises from about 0.1% to about 1% by weight of the pharmaceutical formulation.

In some embodiments, the filler/diluent component comprises one or more of mannitol, lactose, sucrose, powdered cellulose, microcrystalline cellulose, maltodextrin, sorbitol, starch, xylitol, carboxymethyl cellulose, carboxyethyl cellulose, hydroxyethyl cellulose, starch, a calcium phosphate, for example anhydrous dicalcium phosphate, sodium starch glycolate, or metal aluminosilicate, for example, magnesium aluminometasilicate (Neusilin®). In some embodiments, the filler/diluent component comprises mannitol, for example, Pearlitol® 200D.

In some embodiments, the optional second filler/diluent component comprises one or more of mannitol, lactose, sucrose, powdered cellulose, microcrystalline cellulose, maltodextrin, sorbitol, starch, xylitol, carboxymethyl cellulose, carboxyethyl cellulose, hydroxyethyl cellulose, starch, a calcium phosphate, for example, anhydrous dicalcium phosphate, sodium starch glycolate, or metal aluminosilicate, for example, magnesium aluminometasilicate (Neusilin®). In some embodiments, the optional second filler/diluent component comprises microcrystalline cellulose, for example, Avicel® PH101.

In some embodiments, the surface modifying agent component comprises one or more of Poloxamer 188, metal alkyl sulfate, sodium lauryl sulfate, polyoxyethylene sorbitan fatty acid ester, polyethylene glycol, polyoxyethylene castor oil derivative, docusate sodium, quaternary ammonium amine compound, sugar esters of fatty acid or glycerides of fatty acid. In some embodiments, the surface modifying agent component comprises sodium lauryl sulfate.

In some embodiments, the glidant/disintegrant component comprises one or more of croscarmellose sodium, modified cellulose, pregelatinized starch, sodium starch glycolate, crospovidone, starch, alginic acid, sodium alginate, clay, cellulose floc, ion exchange resin, effervescent systems based on food acids and an alkaline component, silica such as Aerosil® 200, talc, lactose, stearate, dibasic calcium phosphate, magnesium carbonate, magnesium oxide, calcium silicate, silicon dioxide, or silicon dioxide aerogel. In some embodiments, the glidant/disintegrant component comprises silica, for example, Aerosil® 200.

In some embodiments, the lubricant component comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica such as Aerosil® 200, and sodium chloride. In some embodiments, the lubricant component comprises magnesium stearate.

In some embodiments, the filler/diluent component comprises one or more of mannitol, lactose, sucrose, powdered cellulose, microcrystalline cellulose, maltodextrin, sorbitol, starch, xylitol or a metal aluminosilicate; the optional second filler/diluent component comprises one or more of mannitol, lactose, sucrose, powdered cellulose, microcrystalline cellulose, maltodextrin, sorbitol, starch, xylitol or a metal aluminosilicate; the surface modifying agent component comprises one or more of Poloxamer 188, metal alkyl sulfate, sodium lauryl sulfate, or polyethylene glycol; the glidant/disintegrant component comprises one or more of croscarmellose sodium, modified cellulose, pregelatinized starch, sodium starch glycolate, crospovidone, starch, alginic acid, sodium alginate, silica such as Aerosil® 200, lactose, stearate, dibasic calcium phosphate, magnesium carbonate, magnesium oxide, calcium silicate or silicon dioxide; and the lubricant component comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica such as Aerosil® 200, or sodium chloride.

In some further embodiments, the filler/diluent component comprises mannitol; the optional second filler/diluent component comprises microcrystalline cellulose; the surface modifying agent component comprises sodium lauryl sulfate; the glidant/disintegrant component comprises silica; and the lubricant component comprises a metallic stearate. In some still further embodiments, the filler/diluent component comprises Pearlitol® 200SD; the optional second filler/diluent component comprises Avicel® PH101; the surface modifying agent component comprises sodium lauryl sulfate; the glidant/disintegrant component comprises Aerosil 200; and the lubricant component comprises magnesium stearate.

In some embodiments of the processes and formulations of the invention, the pharmaceutical formulation contains from about 1 mg to about 125 mg of active pharmacological agent, or from about 1 mg to about 3 mg of active pharmacological agent; or from about 3 mg to about 7 mg of active pharmacological agent; or from about 20 mg to about 30 mg of active pharmacological agent; or from about 40 mg to about 60 mg of active pharmacological agent; or from about 70 mg to about 80 mg of active pharmacological agent; or from about 90 mg to about 110 mg of active pharmacological agent.

The present invention also provides processes for preparing a pharmaceutical formulation comprising a pharmaceutically effective amount of an active pharmacological agent and a carrier or excipient system, the carrier or excipient system comprising:

a) a filler/diluent component comprising from about 10% to about 60% by weight of the pharmaceutical formulation;

b) a surface modifying agent component comprising from about 1% to about 20% by weight of the pharmaceutical formulation;

c) a glidant/disintegrant component comprising from about 0.01% to about 10% by weight of the pharmaceutical formulation;

d) an optional second filler/diluent component comprising up to about 20% by weight of the pharmaceutical formulation; and

e) a lubricant component comprising up to about 10% by weight of the pharmaceutical formulation;

the process comprising:

i) blending the glidant/disintegrant component and the active pharmacological agent to form a first mixture;

ii) blending the first mixture with the second filler/diluent component to form a second mixture;

iii) mixing the first filler/diluent component and the surface modifying agent component together with the second mixture to form a third mixture; and

iv) mixing the lubricant component with the third mixture to form a final blend; wherein the active pharmacological agent is 2-(3-fluoro-4-hydroxyphenyl)-7-vinyl-1,3-benzoxazol-5-ol or a pharmaceutically acceptable salt thereof. In some embodiments, the pharmacological active agent is micronised.

### DETAILED DESCRIPTION

In some embodiments, the present invention provides pharmaceutical formulations comprising a pharmaceutically effective amount of an active pharmacological agent and a carrier or excipient system, the carrier or excipient system comprising:

a) a filler/diluent component comprising from about 10% to about 60% by weight of the pharmaceutical formulation;

b) a surface modifying agent component comprising from about 1% to about 20% by weight of the pharmaceutical formulation;

c) a glidant/disintegrant component comprising from about 0.01% to about 10% by weight of the pharmaceutical formulation;

d) an optional second filler/diluent component comprising up to about 20% by weight of the pharmaceutical formulation; and

e) a lubricant component comprising up to about 10% by weight of the pharmaceutical formulation;
wherein the active pharmacological agent has the Formula l: 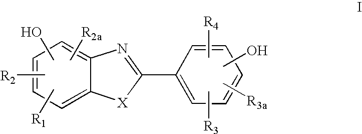
wherein
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅, R₆ are each, independently hydrogen, alkyl of 1-6 carbon atoms, or aryl of 6-10 carbon atoms;
X is O, S, or N R₇; and
R₇ is hydrogen, alkyl of 1-6 carbon atoms, aryl of 6-10 carbon atoms, —COR₅, —CO₂R₅ or —SO₂R₅;
   or a pharmaceutically acceptable salt thereof.

In some embodiments, X is O. In some further embodiments, R₇ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆. In some preferred embodiments, the active ingredient is 2-(3-fluoro-4-hydroxyphenyl)-7-vinyl-1,3-benzoxazol-5-ol or a pharmaceutically acceptable salt thereof.

Generally, the active pharmacological agent comprises up to about 88% by weight of the pharmaceutical formulation. In some embodiments, the active pharmacological agent can be present in an amount of from about 10% to about 50% by weight of the pharmaceutical formulation; from about 20% to about 40% by weight of the pharmaceutical formulation; or from about 25% to about 35% by weight of the pharmaceutical formulation. Preferably, the active pharmacological agent is 2-(3-fluoro-4-hydroxyphenyl)-7-vinyl-1,3-benzoxazol-5-ol or a pharmaceutically acceptable salt thereof.

Generally, the filler/diluent is present in an amount of about 10% to about 60% by weight of the pharmaceutical formulation, about 30% to about 60% by weight of the pharmaceutical formulation, about 40% to about 60% by weight of the pharmaceutical formulation, or about 44% to about 53% by weight of the pharmaceutical formulation. The optional second filler/diluent component is generally present in an amount of up to about 20% by weight of the pharmaceutical formulation, from about 10% to about 20% by weight of the pharmaceutical formulation, or about 12% to about 18% by weight of the pharmaceutical formulation. In some embodiments, the filler/diluent component and the second filler/diluent component include one or more agent that is useful as a filler or diluent or a combination of such agents. Both the filler/diluent and the second filler/diluent can be selected from fillers and diluents known to.be useful in the art, including for example, mannitol, lactose, sucrose, powdered cellulose, microcrystalline cellulose, maltodextrin, sorbitol, starch, xylitol, carboxymethyl cellulose, carboxyethyl cellulose, hydroxyethyl celluloses, starches, calcium phosphates, for example, anhydrous dicalcium phosphate, sodium starch glycolates, and metal aluminosilicates, for example, magnesium aluminometasilicate (Neusilin®). One or more fillers and/or one or more diluents may be selected in each case. In some embodiments, the filler/diluent component comprises mannitol, for example, Pearlitol® 200D, and the second filler/diluent comprises microcrystalline cellulose, for example, Avicel® PH101.

Generally, the surface modifying agent component is present in an amount of from about 1% to about 20% by weight of the pharmaceutical formulation; about 1% to about 10% by weight of the pharmaceutical formulation, about 3% to about 7% by weight of the pharmaceutical formulation, or about 4% to about 6% by weight of the pharmaceutical formulation. The surface modifying agent can be selected from surface modifying agents, known to be useful in the art, including, for example, surfactants, Poloxamer 188, metal alkyl sulfates such as sodium lauryl sulfate, polyoxyethylene sorbitan fatty acid esters, polyethylene glycols, polyoxyethylene castor oil derivatives, docusate sodium, quaternary ammonium amine compounds, sugar esters of fatty acids and glycerides of fatty acids. In some embodiments, the surface modifying agent component comprises sodium lauryl sulfate.

Generally, the glidant/disintegrant component is present in an amount from about 0.01% to about 10% by weight of the pharmaceutical formulation, about 0.01% to about 5% by weight of the pharmaceutical formulation, or about 1% to about 2% by weight of the pharmaceutical formulation. The glidant/diluent can be selected from glidants and disintegrants known to be useful for pharmaceutical formulations. One or more glidants and/or one or more disintegrants may be selected. Examples of suitable glidant/disintegrants include croscarmellose sodium, modified cellulose, pregelatinized starch, sodium starch glycolate, crospovidone, starch, alginic acid, sodium alginate, clays, cellulose floc, ion exchange resins, effervescent systems based on food acids and an alkaline carbonate component, silica such as Aerosil® 200, talc, lactose, stearates, dibasic calcium phosphate, magnesium carbonate, magnesium oxide, calcium silicate, silicon dioxide, and silicon dioxide aerogels. In some embodiments, the glidant/diluent is a silica, for example, Aerosil® 200. The glidant/disintegrant component is preferably an agent that is useful both as a glidant and as a disintegrant or a combination of such agents.

The lubricant component is present in an amount of up to about 10% of the formulation, from about 0.01% to about 2% of the formulation, from about 0.01% to about 1% of the formulation, or from about 0.1% to about 1% of the formulation. The lubricant can be selected from the many lubricants useful in the pharmaceutical arts. Examples of suitable lubricants include metallic stearates, fatty acid esters, fatty acids, fatty alcohols, glyceryl behenate, mineral oil, paraffins, hydrogenated vegetable oils, leucine, polyethylene glycols, metallic lauryl sulfates, silica such as Aerosil® 200, and sodium chloride. In some embodiments, the lubricant is magnesium stearate.

Additional suitable filler/diluents, surface modifying agents, glidant/disintegrants and lubricants can be found in, for example, Remington's Pharmaceutical Sciences, 17th ed., Mack Publishing Company, Easton, Pa., 1985, which is incorporated herein by reference in its entirety.

In some preferred embodiments, the formulation contains from about 1 mg to about 125 mg, or about 1 mg to about 3 mg, or about 3 mg to about 7 mg, or about 20 mg to about 30 mg, or about 40 mg to about 60 mg, or about 70 mg to about 80 mg, or about 90 mg to about 110 mg of active pharmacological agent.

The present invention also provides processes for preparing a pharmaceutical formulation comprising a pharmaceutically effective amount of an active pharmacological agent and a carrier or excipient system, the carrier or excipient system comprising:

a) a filler/diluent component comprising from about 10% to about 60% by weight of the pharmaceutical formulation;

b) a surface modifying agent component comprising from about 1% to about 20% by weight of the pharmaceutical formulation;

c) a glidant/disintegrant component comprising from about 0.01% to about 10% by weight of the pharmaceutical formulation;

d) an optional second filler/diluent component comprising up to about 20% by weight of the pharmaceutical formulation; and

e) a lubricant component comprising up to about 10% by weight of the pharmaceutical formulation;

the process comprising:

i) blending the glidant/disintegrant component and the active pharmacological agent to form a first mixture;

ii) blending the first mixture with the second filler/diluent component to form a second mixture;

iii) mixing the first filler/diluent component and the surface modifying agent component together with the second mixture to form a third mixture; and

iv) mixing the lubricant component with the third mixture to form a final blend; wherein the active pharmacological agent is 2-(3-fluoro4-hydroxyphenyl)-7-vinyl-1,3-benzoxazol-5-ol or a pharmaceutically acceptable salt thereof.

In some embodiments, the processes further comprise encapsulating at least a portion of the final blend.

The present invention also provides products of the processes described herein.

It will be understood that the weight percentages set forth for the filler/diluent component, surface modifying agent component, disintegrant component, optional second filler component, and lubricant component of the formulations disclosed herein are the percentages that each component will comprise of a final pharmaceutical formulation, without reference to any surface covering, such as a tablet coating or capsule. The remainder of the final formulation will be comprised of the active pharmacological agent(s).

Oral formulations containing the present solid dispersions can comprise any conventionally used oral forms, including tablets, capsules, buccal forms, troches, lozenges and oral liquids, suspensions, and the like. Capsules are preferred. Capsules or tablets containing the present solid dispersion can also be combined with mixtures of other active compounds or inert fillers/diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc. In some preferred embodiments, the formulations are direct blend solid dispersions contained in capsules.

Tablet formulations can be made by conventional compression, wet granulation, or dry granulation methods and utilize pharmaceutically acceptable fillers/diluents, binding agents, lubricants, disintegrants, suspending or stabilizing agents, including, but not limited to, magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Oral formulations used herein may utilize standard delay or time release formulations or spansules. Suppository formulations may be made from traditional materials, including cocoa butter, with or without the addition of waxes to alter the suppositories melting point, and glycerin. Water soluble suppository bases, such as polyethylene glycols of various molecular weights, may also be used.

Film coatings useful with the present formulations are known in the art and generally consist of a polymer (usually a cellulosic type of polymer), a colorant and a plasticizer. Additional ingredients such as wetting agents, sugars, flavors, oils and lubricants can be included in film coating formulations to impart certain characteristics to the film coat. The formulations and formulations herein may also be combined and processed as a solid, then placed in a capsule form such as a gelatin capsule.

As will be appreciated, some components of the formulations of the invention can possess multiple functions. For example, a given component can act as both a filler/diluent and a disintegrant. In some such cases, the function of a given component can be considered singular even though its properties may allow multiple functionality.

The pharmaceutical formulations and excipient systems herein can also contain an antioxidant or a mixture of antioxidants such as ascorbic acid. Other antioxidants that can be used include sodium ascorbate and ascorbyl palmitate, optionally in conjunction with an amount of ascorbic acid. An example range for the antioxidant(s) is from about up to about 15% by weight, e.g., from about 0.05% to about 15% by weight, from about 0.5% to about 15% by weight, or from about 0.5% to about 5% by weight. In some embodiments, the pharmaceutical formulations contain substantially no antioxidant.

Additional numerous various excipients, dosage forms, dispersing agents and the like that are suitable for use in connection with the solid dispersions of the invention are known in the art and described in, for example, Remington's Pharmaceutical Sciences, 17th ed., Mack Publishing Company, Easton, Pa., 1985, which is incorporated herein by reference in its entirety.

The materials, methods, and examples presented herein are intended to be illustrative, and are not intended to limit the scope of the invention.

### EXAMPLES

### Example 1

### Procedure for Preparation of Capsules Containing ERB-041

Amounts of components are shown in the Table below.
1. Aerosil® 200 and ERB-041 are mixed together for 10 minutes using a tumbling type blender at 30 rpm.
2. The preblend from Step 1 is then passed through a 500 micron screen.
3. Avicel® PH101 is used to wash the internal surfaces of the screen and mixing vessel, and is then passed through a 500 micron screen and blended with the sieved pre-blend from Step 2 for a further 10 minutes.
4. Pearlitol® 200SD and sodium lauryl sulfate are passed through a 500 micron screen and mixed with the blend from Step 3 for 10 minutes.
5. Magnesium stearate is mixed with a portion of the blend from Step 4 and the mixture passed through a 500 micron screen and blended with the bulk of the blend from Step 4 for an additional one minute.
6. The final blend is then encapsulated into size 1 propyl hydroxymethyl cellulose (HPMC) capsule shells.

The formulation of the capsules is shown in the Table below.

| Ingredient | % WT/WT | mg/capsule | |
|---|---|---|---|
| ERB-041 Micronised | 30.00 | 100.000 | mg |
| Aerosil ® 200 | 1.70 | 5.667 | mg |
| Avicel ® PH101 | 14.50 | 48.333 | mg |
| Sodium Lauryl Sulfate | 5.00 | 16.667 | mg |
| Pearlitol ® 200SD | 48.30 | 161.000 | mg |
| Magnesium Stearate | 0.50 | 1.667 | mg |
| TOTAL | 100.00 | 333.33 | mg |

It is intended that each of the patents, applications, and printed publications, including books, mentioned in this patent document be hereby incorporated by reference in their entirety.

As those skilled in the art will appreciate, numerous changes and modifications may be made to the embodiments of the invention without departing from the spirit of the invention. It is intended that all such variations fall within the scope of the invention.

What is claimed is:

1. A pharmaceutical formulation comprising a pharmaceutically effective amount of an active pharmacological agent and a carrier or excipient system, the carrier or excipient system comprising:
a) a filler/diluent component comprising from about 10% to about 60% by weight of the pharmaceutical formulation;
b) a surface modifying agent component comprising from about 1% to about 20% by weight of the pharmaceutical formulation;
c) a glidant/disintegrant component comprising from about 0.01% to about 10% by weight of the pharmaceutical formulation;
d) an optional second filler/diluent component comprising up to about 20% by weight of the pharmaceutical formulation; and
e) a lubricant component comprising up to about 10% by weight of the pharmaceutical formulation;
wherein the active pharmacological agent has the Formula I: 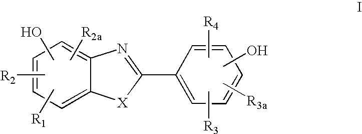
wherein
R₁ is hydrogen, hydroxyl, halogen, alkyl of 1-6 carbon atoms, trifluoroalkyl of 1-6 carbon atoms, cycloalkyl of 3-8 carbon atoms, alkoxy of 1-6 carbon atoms, trifluoroalkoxy of 1-6 carbon atoms, thioalkyl of 1-6 carbon atoms, sulfoxoalkyl of 1-6 carbon atoms, sulfonoalkyl of 1-6 carbon atoms, aryl of 6-10 carbon atoms, a 5 or 6-membered heterocyclic ring having 1 to 4 heteroatoms selected from O, N or S, —NO₂, —NR₅R₆, —N(R₅)COR₆, —CN, —CHFCN, —CF₂CN, alkynyl of 2-7 carbon atoms, or alkenyl of 2-7 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₂ and R₂ₐ are each, independently, hydrogen, hydroxyl, halogen, alkyl of 1-6 carbon atoms, alkoxy of 1-4 carbon atoms, alkenyl of 2-7 carbon atoms, alkynyl of 2-7 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₃, R₃ₐ, and R₄ are each, independently, hydrogen, alkyl of 1-6 carbon atoms, alkenyl of 2-7 carbon atoms, alkynyl of 2-7 carbon atoms, halogen, alkoxy of 1-4 carbon atoms, trifluoroalkyl of 1-6 carbon atoms, or trifluoroalkoxy of 1-6 carbon atoms; wherein the alkyl or alkenyl moieties are optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆;
R₅ and R₆ are each, independently hydrogen, alkyl of 1-6 carbon atoms, or aryl of 6-10 carbon atoms;
X is O, S, or N R₇; and
R₇ is hydrogen, alkyl of 1-6 carbon atoms, aryl of 6-10 carbon atoms, —COR₅, —CO₂R₅ or —SO₂R₅; or
a pharmaceutically acceptable salt thereof.

2. The pharmaceutical formulation of claim 1 wherein X is O.

3. The pharmaceutical formulation of claim 2, wherein R₁ is alkenyl of 2-3 carbon atoms, which is optionally substituted with hydroxyl, —CN, halogen, trifluoroalkyl, trifluoroalkoxy, —COR₅, —CO₂R₅, —NO₂, CONR₅R₆, NR₅R₆ or N(R₅)COR₆.

4. The pharmaceutical formulation of claim 1 wherein the active ingredient is 2-(3-fluoro-4-hydroxyphenyl)-7-vinyl-1,3-benzoxazol-5-ol or a pharmaceutically acceptable salt thereof.

5. The pharmaceutical formulation of claim 1 or claim 4 wherein the active pharmacological agent comprises up to about 88% by weight of the pharmaceutical formulation.

6. The pharmaceutical formulation of claim 1 or claim 4 wherein:
the active pharmacological agent comprises from about 10% to about 50% by weight of the pharmaceutical formulation;
the filler/diluent component comprises from about 30% to about 60% by weight of the pharmaceutical formulation;
the surface modifying agent component comprises from about 1% to about 10% by weight of the pharmaceutical formulation;
the glidant/disintegrant component comprises from about 0.01% to about 5% by weight of the pharmaceutical formulation;
the optional second filler/diluent component comprises from about 10% to about 20% by weight of the pharmaceutical formulation; and
the lubricant component comprises from about 0.01% to about 2% by weight of the pharmaceutical formulation.

7. The pharmaceutical formulation of claim 1 or claim 4 wherein:
the active pharmacological agent comprises from about 20% to about 40% by weight of the pharmaceutical formulation;
the filler/diluent component comprises from about 40% to about 60% by weight of the pharmaceutical formulation;
the surface modifying agent component comprises from about 3% to about 7% by weight of the pharmaceutical formulation;
the glidant/disintegrant component comprises from about 1% to about 2% by weight of the pharmaceutical formulation;
the optional second filler/diluent component comprises from about 10% to about 20% by weight of the pharmaceutical formulation; and
the lubricant component comprises from about 0.01% to about 1% by weight of the pharmaceutical formulation.

8. The pharmaceutical formulation of claim 1 or claim 4 wherein:
the active pharmacological agent comprises from about 25% to about 35% by weight of the pharmaceutical formulation;
the filler/diluent component comprises from about 44% to about 53% by weight of the pharmaceutical formulation;
the surface modifying agent component comprises from about 4% to about 6% by weight of the pharmaceutical formulation;
the glidant/disintegrant component comprises from about 1% to about 2% by weight of the pharmaceutical formulation;
the optional second filler/diluent component comprises from about 12% to about 18% by weight of the pharmaceutical formulation; and
the lubricant component comprises from about 0.1% to about 1% by weight of the pharmaceutical formulation

9. The pharmaceutical formulation of claim 1 or claim 4 wherein the filler/diluent component comprises one or more of mannitol, lactose, sucrose, powdered cellulose, microcrystalline cellulose, maltodextrin, sorbitol, starch, xylitol, carboxymethyl cellulose, carboxyethyl cellulose, hydroxyethyl cellulose, starch, a calcium phosphate, anhydrous dicalcium phosphate, sodium starch glycolate, metal aluminosilicate, or magnesium aluminometasilicate.

10. The pharmaceutical formulation of claim 1 or claim 4 wherein the filler/diluent component comprises mannitol.

11. The pharmaceutical formulation of claim 1 or claim 4 wherein the optional second filler/diluent component comprises one or more of mannitol, lactose, sucrose, powdered cellulose, microcrystalline cellulose, maltodextrin, sorbitol, starch, xylitol, carboxymethyl cellulose, carboxyethyl cellulose, hydroxyethyl cellulose, starch, a calcium phosphate, anhydrous dicalcium phosphate, sodium starch glycolate, metal aluminosilicate, or magnesium aluminometasilicate.

12. The pharmaceutical formulation of claim 1 or claim 4 wherein the optional second filler/diluent component comprises microcrystalline cellulose.

13. The pharmaceutical formulation of claim 1 or claim 4 wherein the surface modifying agent component comprises one or more of Poloxamer 188, metal alkyl sulfate, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, or glyceride of fatty acid.

14. The pharmaceutical formulation of claim 1 or claim 4 wherein the surface modifying agent component comprises sodium lauryl sulfate.

15. The pharmaceutical formulation of claim 1 or claim 4 wherein the glidant/disintegrant component comprises one or more of croscarmellose sodium, modified cellulose, pregelatinized starch, sodium starch glycolate, crospovidone, starch, alginic acid, sodium alginate, clay, cellulose floc, ion exchange resin, effervescent system based on food acids and an alkaline carbonate component, talc, lactose, stearate, dibasic calcium phosphate, magnesium carbonate, magnesium oxide, calcium silicate, silica, silicon dioxide, or silicon dioxide aerogel.

16. The pharmaceutical formulation of claim 1 or claim 4 wherein the glidant/disintegrant component comprises silica.

17. The pharmaceutical formulation of claim 1 or claim 4 wherein the lubricant component comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica, or sodium chloride.

18. The pharmaceutical formulation of claim 1 or claim 4 wherein the lubricant component comprises magnesium stearate.

19. The pharmaceutical formulation of claim 1 or claim 4 wherein:
the filler/diluent component comprises one or more of mannitol, lactose, sucrose, powdered cellulose, microcrystalline cellulose, maltodextrin, sorbitol, starch, xylitol, a metal aluminosilicate, or magnesium aluminometasilicate;
the optional second filler/diluent component comprises one or more of mannitol, lactose, sucrose, powdered cellulose, microcrystalline cellulose, maltodextrin, sorbitol, starch, xylitol, a metal aluminosilicate, or magnesium aluminometasilicate;
the surface modifying agent component comprises one or more of Poloxamer 188, metal alkyl sulfate, sodium lauryl sulfate, or polyethylene glycol;
the glidant/disintegrant component comprises one or more of croscarmellose sodium, modified cellulose, pregelatinized starch, sodium starch glycolate, crospovidone, starch, alginic acid, sodium alginate, silica, lactose, stearate, dibasic calcium phosphate, magnesium carbonate, magnesium oxide, calcium silicate, or silicon dioxide; and
the lubricant component comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica, or sodium chloride.

20. The pharmaceutical formulation of claim 1 or claim 4 wherein:
the filler/diluent component comprises mannitol;
the optional second filler/diluent component comprises microcrystalline cellulose;
the surface modifying agent component comprises sodium lauryl sulfate;
the glidant/disintegrant component comprises silica; and
the lubricant component comprises a metallic stearate.

21. The pharmaceutical formulation of claim 1 wherein the formulation contains from about 1 mg to about 125 mg of active pharmacological agent.

22. The pharmaceutical formulation of claim 1 wherein the formulation contains from about 1 mg to about 3 mg of active pharmacological agent.

23. The pharmaceutical formulation of claim 1 wherein the formulation contains from about 3 mg to about 7 mg of active pharmacological agent.

24. The pharmaceutical formulation of claim 1 wherein the formulation contains from about 20 mg to about 30 mg of active pharmacological agent.

25. The pharmaceutical formulation of claim 1 wherein the formulation contains from about 40 mg to about 60 mg of active pharmacological agent.

26. The pharmaceutical formulation of claim 1 wherein the formulation contains from about 70 mg to about 80 mg of active pharmacological agent.

27. The pharmaceutical formulation of claim 1 wherein the formulation contains from about 90 mg to about 110 mg of active pharmacological agent.

28. The pharmaceutical formulation of claim 4 wherein the formulation contains from about 1 mg to about 125 mg of active pharmacological agent.

29. The pharmaceutical formulation of claim 4 wherein the formulation contains from about 1 mg to about 3 mg of active pharmacological agent.

30. The pharmaceutical formulation of claim 4 wherein the formulation contains from about 3 mg to about 7 mg of active pharmacological agent.

31. The pharmaceutical formulation of claim 4 wherein the formulation contains from about 20 mg to about 30 mg of active pharmacological agent.

32. The pharmaceutical formulation of claim 4 wherein the formulation contains from about 40 mg to about 60 mg of active pharmacological agent.

33. The pharmaceutical formulation of claim 4 wherein the formulation contains from about 70 mg to about 80 mg of active pharmacological agent.

34. The pharmaceutical formulation of claim 4 wherein the formulation contains from about 90 mg to about 110 mg of active pharmacological agent.

35. A process for preparing a pharmaceutical formulation comprising a pharmaceutically effective amount of an active pharmacological agent and a carrier or excipient system, the carrier or excipient system comprising:
a) a filler/diluent component comprising from about 10% to about 60% by weight of the pharmaceutical formulation;
b) a surface modifying agent component comprising from about 1% to about 20% by weight of the pharmaceutical formulation;
c) a glidant/disintegrant component comprising from about 0.01% to about 10% by weight of the pharmaceutical formulation;
d) an optional second filler/diluent component comprising up to about 20% by weight of the pharmaceutical formulation; and
e) a lubricant component comprising up to about 10% by weight of the pharmaceutical formulation;
the process comprising:
i) blending the glidant/disintegrant component and the active pharmacological agent to form a first mixture;
ii) blending the first mixture with the second filler/diluent component to form a second mixture;
iii) mixing the first filler/diluent component and the surface modifying agent component together with the second mixture to form a third mixture; and
iv) mixing the lubricant component with the third mixture to form a final blend;
wherein the active pharmacological agent is 2-(3-fluoro-4-hydroxyphenyl)-7-vinyl-1,3-benzoxazol-5-ol or a pharmaceutically acceptable salt thereof.

36. The process of claim 35 wherein the pharmacological active agent is micronised.

37. The process of claim 35 wherein:
the filler/diluent component comprises one or more of mannitol, lactose, sucrose, powdered cellulose, microcrystalline cellulose, maltodextrin, sorbitol, starch, xylitol, carboxymethyl cellulose, carboxyethyl cellulose, hydroxyethyl cellulose, starch, a calcium phosphate, anhydrous dicalcium phosphate, sodium starch glycolate, metal aluminosilicate, or magnesium aluminometasilicate;
the optional second filler/diluent component comprises one or more of mannitol, lactose, sucrose, powdered cellulose, microcrystalline cellulose, maltodextrin, sorbitol, starch, xylitol, carboxymethyl cellulose, carboxyethyl cellulose, hydroxyethyl cellulose, starch, a calcium phosphate, anhydrous dicalcium phosphate, sodium starch glycolate, metal aluminosilicate, or magnesium aluminometasilicate;
the surface modifying agent component comprises one or more of Poloxamer 188, metal alkyl sulfate, sodium lauryl sulfate, polyoxyethylene sorbitan fatty acid ester, polyethylene glycol, polyoxyethylene castor oil derivative, docusate sodium, quaternary ammonium amine compound, sugar ester of fatty acid, or glyceride of fatty acid;
the glidant/disintegrant component comprises one or more of croscarmellose sodium, modified cellulose, pregelatinized starch, sodium starch glycolate, crospovidone, starch, alginic acid, sodium alginate, clay, cellulose floc, ion exchange resin, effervescent system based on food acids and an alkaline carbonate component, talc, lactose, stearate, dibasic calcium phosphate, magnesium carbonate, magnesium oxide, calcium silicate, silica, silicon dioxide, or silicon dioxide aerogel; and
the lubricant component comprises one or more of metallic stearate, fatty acid ester, fatty acid, fatty alcohol, glyceryl behenate, mineral oil, paraffin, hydrogenated vegetable oil, leucine, polyethylene glycol, metallic lauryl sulfate, silica, or sodium chloride.

38. The process of claim 35 wherein:
the filler/diluent component comprises mannitol;
the optional second filler/diluent component comprises microcrystalline cellulose;
the surface modifying agent component comprises sodium lauryl sulfate;
the glidant/disintegrant component comprises silica; and
the lubricant component comprises a metallic stearate.

39. The process of claim 35 wherein the formulation contains from about 1 mg to about 125 mg of active pharmacological agent.

40. The process of claim 35 wherein the formulation contains from about 1 mg to about 3 mg of active pharmacological agent.

41. The process of claim 35 wherein the formulation contains from about 3 mg to about 7 mg of active pharmacological agent.

42. The process of claim 35 wherein the formulation contains from about 20 mg to about 30 mg of active pharmacological agent.

43. The process of claim 35 wherein the formulation contains from about 70 mg to about 80 mg of active pharmacological agent.

44. The process of claim 35 wherein the formulation contains from about 90 mg to about 110 mg of active pharmacological agent.

45. A product of the process of any of claims 35-44.

46. A capsule or tablet comprising a pharmaceutical formulation of claim 1.
